(19) United States
(12) Patent Application Publication
(10) Pub. No.: US 2005/0090715 A1
(43) Pub. Date: Apr. 28, 2005

(54) Spatula device for performance of laryngoscopical treatment

(76) Inventor: Armin Schorer, Irndorf (DE)

Correspondence Address: CLARK & ELBING LLP, 101 FEDERAL STREET, BOSTON, MA, 02110, US

(21) Appl. No.: 10/693,400

(22) Filed: Oct. 24, 2003

(51) Int. Cl. A61B 1/267

(52) U.S. Cl. 600/188; 600/193

(57) ABSTRACT

A Spatula device for performance of a laryngoscopical treatment of patients which are intubatable conventionally or with difficulties characterized in that the spatula device comprises a flat element at its distal end wherein the flat element is hinged.

### FIELD OF THE INVENTION

This invention relates generally to a spatula device for performance of laryngoscopical treatment, and more particularly to a spatula device for performance of laryngoscopical treatment wherein the distal end of the spatula device is hinged.

### BACKGROUND OF THE INVENTION

Performance of a laryngoscopical treatment requires the usage of a spatula device which is inserted in the mouth of a patient such that the patient's tongue is displaced and a patient's larynx with epiglottis and vocal cords is made visible for inspection purposes only or for performance of an intubation treatment afterwards. Prior art spatula devices are designed having a substantially curved shape (McIntosh spatula device) or a substantially straight shape (Miller spatula device) and are provided with a light source for lightening the area to be inspected. Prior art spatula devices may be sufficient; however, problems occur in cases where the pharynx of a patient is hard or difficult to inspect. This situation is apparent, e.g., if a patient can only offer a little jaw mobility, little mobility of cervical spinal column, if a tumor in the area of upper airway exists, or if there is significant danger for injury of anterior teeth. In such a situation a laryngoscopical treatment is performed with a significant risk for injuring the patient. In case the risk is qualified as being too high, laryngoscopical treatment is interrupted or avoided totally. If there is enough time available for qualifying the risk of a laryngoscopical treatment, e.g., X-ray inspection is performed before. However, in emergency cases there is often no possibility for a serious inspection before laryngoscopical treatment or intubation treatment so that an operator has to work with a risk that might be combined with severe consequences such as injuring patient's trachea opening or interruption of oxygen breathing with a consequence of brain injury. In “Der Anaesthesist”, 1996, vol. 45, 12, pages 1248-1267, some methods are disclosed showing how even in difficult cases, a trachea opening might be observed, and an intubation might be performed with less risk. These methods, however, afford an enormous effort with respect to apparatuses and practical experience.

Therefore, there is a need for a spatula device enabling a laryngoscopical treatment in usual cases as well as in cases where a patient's pharynx is hard or difficult to inspect. Furthermore, it would be desirable if such a spatula device would be easy to handle, would need only a short teaching phase, and could be used with small effort with respect to apparatuses and practical experience even in emergency cases.

### SUMMARY OF THE INVENTION

According to the present invention the objects of the present invention are achieved by the features of the independent claim 1. Advantageous further embodiments are described in the subclaims.

### BRIEF DESCRIPTION OF THE DRAWINGS

The above and other objects, features and advantages of the present invention will be more apparent from the following detailed description taken in conjunction with the accompanying drawings in which:

FIG. 1 is a side view of a spatula device in accordance with the present invention;

FIG. 2 is a top view of a spatula device in accordance with the present invention.

### DETAILED DESCRIPTION OF PREFERRED EMBODIMENT

Hereinafter, a preferred embodiment of the present invention will be described with reference to the accompanying drawings. In the following description and drawings the same reference numerals are used to designate the same components and so repetition of the description of the same components will be omitted.

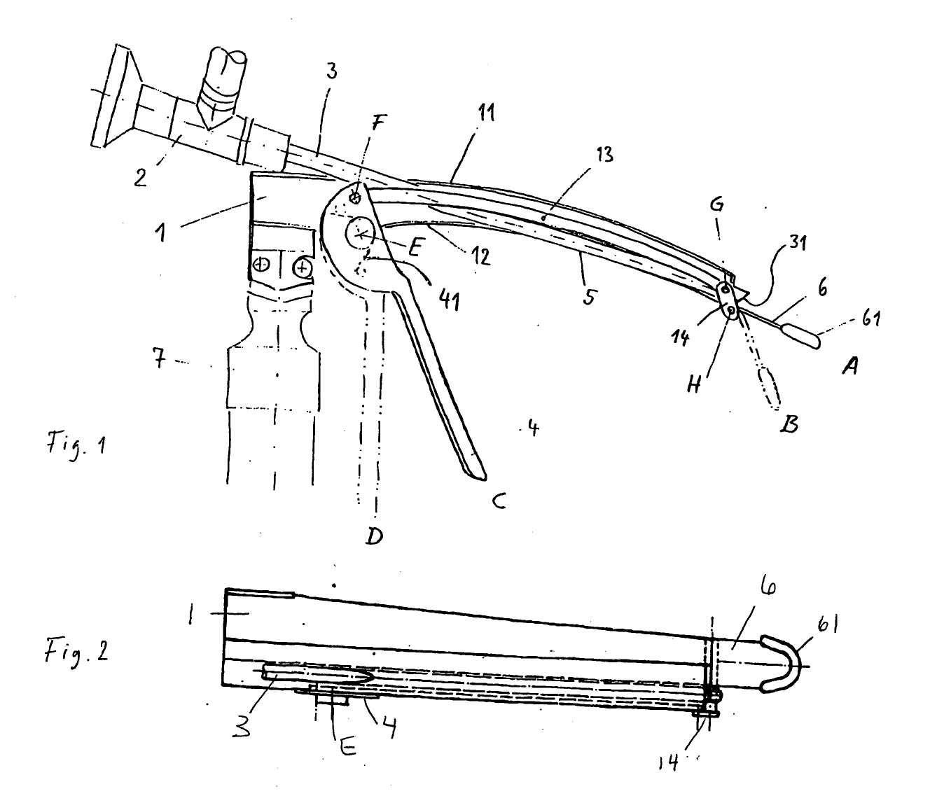

FIG. 1 shows a spatula device 1 in accordance with the present invention. The spatula device 1 is connected at its one end to a grip 7. The spatula device 1 is connected such that it can be exchanged easily from the grip 7. This is advantageous since only one grip is necessary in order to use spatula devices of different sizes.

The spatula device is provided with a top surface 11 and a bottom surface 12, wherein the bottom surface 12 is brought into direct contact with the patient's tongue during laryngoscopical treatment. Regarded from a side view, see FIG. 1, the top surface 11 and the bottom surface 12 are formed approximately in an arc segment shape according to prior art McIntosh design. However, both surfaces may be formed in a straight line according to Miller design of prior art as well. Due to further elements added to the spatula device, as explained later, the arc segment shape of the spatula device is preferred.

The spatula device may be designed in a disposable type so that a cross-infection from one patient to the next is avoided. A disposable spatula device type is preferably made of plastics material and is economical in usage. However, the spatula device may be made of metal material as well, requiring serious cleaning after each usage.

At the other distal end of the spatula device 1 a flat element 6 is provided wherein this flat element 6 is hinged from a position A towards a position B. In position A the flat element 6 forms an extension of the arc segment portion of the bottom surface 12 of the spatula device 1, wherein the extension may be a tangent or a continuation of the arc segment.

During insertion of the spatula device 1 into patient's mouth said flat element 6 is preferably in position A. In case an operator reaches a final position in patient's mouth and difficulties arise in observing patient's larynx and/or trachea opening the operator may hinge down flat element 6 carefully towards position B. By moving flat element 6 from position A towards position B the operator is enabled to displace a patient's tongue base thereby making the larynx and trachea opening more better observable for the operator. The movement from position A towards position B may be interrupted by the operator if desired and then finished in a position between position A and position B. Preferably, such a movement of flat element 6 is infinitely variable. Therewith, danger of injuring of the patient by moving flat element 6 too far is kept at a low level.

Moving flat element 6 from position A towards position B is performed by means of moving a pulling handle 4 from position C towards position D, see FIG. 1. The pulling handle 4 is pivoted in axis E and connected with spatula device 1 (not shown). By means of a connection pin pivoted in axis F a connection is provided from pulling handle 4 to one end of a first coupling element 13. The other end of the first coupling element 13 is connected with a connection pin pivoted in axis G wherein a connection is provided between first coupling element 13 and one end of second coupling element 14. Furthermore, the other end of second coupling element 14 is connected with connection pin pivoted in axis H, connected with spatula device 1. Flat element 6 is firmly fixed with the connection pin pivoted in axis H so that by moving pulling handle 4 from position C towards position D flat element 6 is moved from position A towards position B via first coupling element 13 and second coupling element 14.

Preferably, position A of flat element 6 is a normal position and position B of flat element 6 is a hinged position. This is achieved by means of pulling handle 4 being in a normal position in position C and in a hinged position in position D, wherein pulling handle 4 is held in resting position or normal position C by means of a helical torsion spring 41.

The spatula device 1 is provided with an optical device 3, see FIG. 1. The optical device 3 is set through an opening of the top surface 11 of the spatula device 1 until a position of distal end of spatula device 1 is reached where flat element 6 is connected with connection pin pivoted in axis H. As illustrated in FIG. 1, the optical device 3 comprises a stiff pipe 5 and is provided with light transferring fibers. A cold light source can be connected with the optical device 3 by means of an adapter 2 so that by means of fibers an illumination of a space around distal end of spatula device 1 is allowed.

The optical device 3 is provided with a light emission output 31 at its distal end, wherein the light emission output is inclined to longitudinal axis of optical device 3 and is in proximity of the distal end of spatula device 1 so that light is directed towards an area covered by a space defined between position A and position B of flat element 6. By means of an inclined light emission output 31 not only an illumination of a straight extension of the bottom surface 12 of spatula device 1 is allowed but an illumination towards position B of flat element 6 as well. In this respect it is useful that the optical device 3 is provided with a wide-angle lens covering a range of an angle from 0 to 360 degrees, preferably from 0 to 75 degrees, so that sufficient illumination of patient's larynx is made possible.

In contrary to a stiff optical device, the optical device 3 may be flexible with fibers being integrated in the spatula device 1, e.g., between top surface 11 and bottom surface 12. In this case a light source could be integrated in grip 7 wherein the light source would feed the flexible optical device. In general, by means of the optical device 3, either stiff or flexible, observation of patient's larynx is improved.

It is recommended that the optical device 3 is firmly fixed with the spatula device 1. This is advantageous since the operator may then draw full attention on insertion the spatula device 1 in the patient's mouth, moving flat element 6 as desired, observing the patient's larynx and trachea opening and possibly performing an intubation treatment without any help by an assistant illuminating the zone of interest, especially in emergency cases. However, in case of less space available and sufficient light intensity for performing laryngoscopical treatment or intubation the optical device 3 is designed as being removable so that handling of the spatula device 1 is simplified.

As illustrated in FIGS. 1 and 2, distal end of flat element 6 is provided with a thickened rounded lip 61. This may be achieved by means of an additional element connected with flat element 6 or element 61 is integrally formed with flat element 6. Said element 61 having a rounded lip is advantageous due to its atraumatic shape so that an injuring of the patient during moving flat element 6 and therewith pressing apart patient's tongue base is prevented.

Therewith, in case a patient's larynx and trachea opening is difficult to observe by means of prior art spatula devices, a spatula device according to the present invention still allows a laryngoscopical treatment wherein the risk of injuring the patient concerned is reduced. Further, a spatula device according to the present invention is easy to handle and needs only a short teaching phase. In comparison with prior art spatula devices the spatula device according to the present invention is provided with only a few more elements, thereby a high reliability is maintained at low costs. In addition, since the spatula device according to the present invention compared with prior art spatula device is similar in shape and design, there is little practical experience necessary to operate effectively with spatula device according to the present invention, even in emergency cases.

Although a preferred embodiment of the present invention has been described for illustrative purposes those skilled in the art will appreciate that various modifications, additions and substitutions are possible without departing from the scope and spirit of the invention as disclosed in the accompanying claims.

Other embodiments are in the claims.

Reference List for FIGS. 1 and 2
1 spatula device
2 adapter
3 optical device
4 pulling handle
5 pipe
6 flat element
7 grip
11 top surface
12 bottom surface
13 first coupling element
14 second coupling element
31 light emission output
41 helical torsion spring
61 rounded lip
A first position of flat element
B second position of flat element
C first position of pulling handle
D second position of pulling handle
E axis of rotation
F axis of rotation
G axis of rotation
H axis of rotation.

1. A spatula device for performance of a laryngoscopical treatment of patients which are intubatable conventionally or with difficulties comprising:
i) a flat element at its distal end wherein the flat element is hinged, and
ii) a removable optical device for observation of space around the distal end of the spatula device.

2. The spatula device according to claim 1, wherein the distal end is hinged by means of a pulling handle.

3. (canceled).

4. The spatula device according to claim 1, wherein the optical device includes a light emission output, which is inclined to a longitudinal axis of the optical device.

5. The spatula device according to claim 1, wherein the optical device is firmly fixable.

6. (canceled).

7. The spatula device according to claim 1, wherein the optical device is provided in a stiff manner.

8. The spatula device according to claim 1, wherein the optical device is provided in a flexible manner.

9. The spatula device according to claim 1, wherein the optical device includes a wide-angle lens with an angle ranging from 0 to 75 degrees.

10. The spatula device according to claim 1, wherein the optical device comprises a cold light source connection.

11. The spatula device according to claim 1, wherein the flat element comprises a thickened rounded lip.

12. The spatula device according to claim 2, including an optical device for observation of space around distal end of the spatula device.

13. The spatula device according to claim 1, comprising a single flat element at its distal end wherein the single flat element is hinged.

14. The spatula device according to claim 1, wherein the distal end is hinged by means of a pulling handle that is operated in a direction perpendicular to the longitudinal axis of the handle.
